(19) United States
(12) Patent Application Publication
(10) Pub. No.: US 2025/0064515 A1
(43) Pub. Date: Feb. 27, 2025

(54) LOW POWER LASER THERAPY DEVICE WITH UNIFORM INTENSITY DISTRIBUTION

(71) Applicant: LYMA LIFE LIMITED, London (GB)

(72) Inventors: Tamas RÓZSA, Budapest (HU); Lucy Goff, London (GB)

(21) Appl. No.: 18/811,092

(22) Filed: Aug. 21, 2024

(30) Foreign Application Priority Data
Aug. 25, 2023 (EP) 23193535.4
Dec. 21, 2023 (GB) 2319853.4

(51) Int. Cl. A61B 18/20; A61B 18/00

(52) U.S. Cl. CPC A61B 18/20; A61B 2018/00702; A61B 2018/2015; A61B 2018/20553

(57) ABSTRACT

Low power laser therapy device, which comprises at an optical system, the optical system comprising a laser light source, a beam expander in front of the light sources, a raster arrangement of first light scattering elements on the frontal surface of the beam expander, and the device comprise a housing around the optical system having a mouth opening covered by a closing member, wherein in front of the optical system a second light scattering element is arranged that fills the mouth opening, and the beam expander and the first and second light scattering elements are made from transparent material that does not affect the polarization of light rays passing therethrough.

### CROSS-REFERENCE TO RELATED APPLICATION

This application claims priority from EP patent application Ser. No. 23/193,535.4, filed on Aug. 25, 2023, and UK patent application 2319853.4, filed on Dec. 21, 2023, which are herein incorporated by reference in their entirety.

### BACKGROUND

Several laser therapy devices are known which differ from each other on features such as size, type of preferred treatment and whether they have a power supply from the mains or whether the power source is independent from the mains, such as a battery power source.

In the case of devices connected to the mains, special precautions should be made to prevent patients from any possible electrical shock, and such precautions are often implemented by circuits which increase the size and the cost of the devices. Moreover, connecting wires in these circuits often limit the free movement and use.

WO 2017/001876 describes a light therapy device, which uses a beam expander in front of the light source. On a frontal surface of the beam-expander, a raster of small diverting lenses is arranged. In a small distance in front of the beam expander with the diverting lenses thereon, a flat beam expander is arranged made of white, porous polyethylene with a high degree of light attenuation. The reason of using such a light scattering element lies in that the device is intended for the treatment of the human eye being very sensitive to high light intensities and a high attenuation is needed.

This device comprises a long body in which the battery can be arranged and a head portion that provides space for the optical elements.

In EP 3 787 742 B1 a portable, battery-operated laser therapy device is described, in which the available power emitted by the laser light sources is not attenuated and the optical elements are arranged in such a way that their intensities are added in a central zone to increase the penetration depth of the infrared laser light in human tissues. The device uses at least three laser sources with a beam expander and beam scattering system placed in front of the light sources. The device is provided with cooling ribs to dissipate the substantial amount of heat emitted by the plurality of light sources.

Laser therapy may be used at skin depths other than the deep tissues, for example, in the epidermis, dermis or fat layers. In laser therapy, it is advantageous to irradiate large areas of skin with laser light, with the treated area being as large as possible within the power and size constraints of a battery-operated portable device. Irradiating the surface of skin under the device in a uniform way has many benefits, especially for cosmetic purposes.

The object of the invention is to provide a low power laser device which can meet these objectives and can generate substantially uniform intensity within the irradiated area.

### SUMMARY OF THE INVENTION

The device according to the invention is designed as defined in the attached claims.

### BRIEF DESCRIPTION OF THE DRAWINGS

The invention will now be described in connection with preferable embodiments thereof, in which reference will be made to the attached drawings. In the drawing:

FIG. 1 is a sketch showing a light source with beam expander, first beam scattering lenses and second beam scattering lenses;

FIG. 2 is a sketch showing a light source with beam expander, first beam scattering lenses and second beam scattering lenses;

FIG. 3 shows the light intensity distribution of a light source arrangement like that shown in FIG. 2 without the second scattering lens;

FIG. 4 shows the light intensity distribution, wherein the device is similar to that shown in FIG. 2;

FIG. 5 is a plan view taken of a light source arrangement similar to that shown in FIG. 1,

FIG. 6 is another plan view of a light source arrangement similar view to that shown in FIG. 1;

FIG. 7 is an enlarged view of a laser therapy device;

FIG. 8a is a sketch showing a light source with beam expander, first beam scattering lenses, a fish eye lens and second beam scattering lenses;

FIG. 8b is a sketch showing a light source with beam expander, first beam scattering lenses, a fish eye lens and second beam scattering lenses.

### DETAILED DESCRIPTION

The invention relates to a low power laser therapy device, which comprises an optical system, where an optical system comprises a laser light source, a beam expander in front of said light source and a raster arrangement of first light scattering elements on the frontal surface of the associated beam expander. The device additionally comprises a housing around the optical systems that has a mouth opening covered by a closing member.

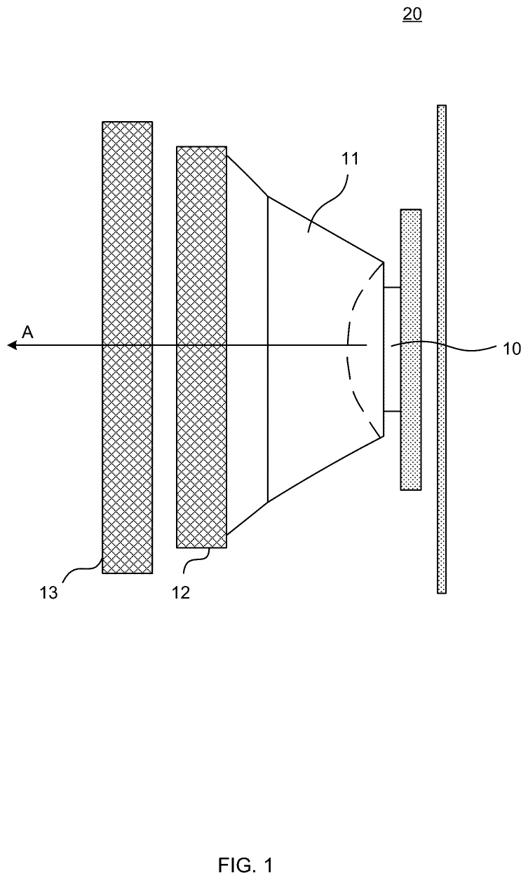

FIG. 1 is an enlarged explanatory sketch of an optical system 20 used in the present invention in front of a single laser light source 10. In front of the light source 10, a beam expander 11 is arranged. The beam expander 11 is made of a light transparent material, such as polymethyl methacrylate, and has a shape designed as a widening cone in forward direction, i.e., along the main direction of light away from the light source 10 and of the axis A of the light source 10. On the frontal surface of the beam expander 11, which is preferably a planar surface and normal to the axis A, first light scattering element 12 is provided.

At a distance in front of the frontal face of the beam expander 11, a second light scattering element 13 is arranged. In various examples, the second light scattering element 13 is made of the same or similar light transparent material as the beam expander 11. However, the second light scattering element 13 need not be made of the same material as the beam expander 11 but it must be light transparent and not changing the polarization of the light passing therethrough. The arrangement of the two light scattering elements 12, 13 within the optical system is referred to herein as a double diffusion system.

In some examples, the light scattering element 12 comprises a plurality of pyramids which are arranged immediately adjacent to each other to form a raster. The second light scattering element 13 has an inner (rear) surface which may also be designed as a raster of pyramids, facing towards the first light scattering elements 12. In this example, the opposing (frontal) surface of the second light scattering element 12 is planar and it is also normal to the axis A.

In other examples, the light scattering elements 12 and 13 comprise a plurality of semi-spheres, frustums and/or swellings. The base/wider portion of the pyramids, semi-spheres, frustum or swellings is the surface of the pyramids, semi-spheres, frustums or swellings which faces the laser light source 10. The pyramids, semi-spheres, frustum or swellings are arranged such that the opposing surface (e.g. the apex of the pyramids, frustums, semi-spheres or swellings) faces the second light scattering element 13.

As another example, the light scattering elements 12 and 13 comprise a frosted diffusion element, such as a frosted diffusion lens. The light scattering elements 12, 13 need not be of the same form. For example, the first light scattering element 12 may comprise pyramids whilst the second light scattering element 13 may comprise a frosted diffusion element etc.

The use of light scattering elements 12, 13 provides the advantage of diffusing the light beam source 10, especially where there are a plurality of light beam sources. Diffusion of the light source is important as it achieves a high attenuation of the light intensity, thereby permitting the laser therapy device to be used in proximity to the eyes.

The use of a single laser light source 10 in a double diffusion system, that is, a system in which there are two opposing light scattering elements 12, 13, at least provides a simplified electronic component layout which may be used to achieve a more uniform intensity of light in a wider treatment area. Therefore, fewer lasers are required to achieve the light intensity required to treat skin.

When light leaves the first light scattering element 12 and enters the surfaces of the second light scattering element 13, there will be minimum attenuation or reflection loss between the first 12 and second 13 scattering elements. Where the scattering elements comprises a plurality of pyramids in a raster arrangement, most of the emitted light will enter normal to the surfaces of the plurality of pyramids on the second light scattering element 13 so that there will be minimum attenuation or reflection loss. To achieve an optimal saving in attenuation or reflection loss, the size of the pyramids and the distance between the two opposite surfaces of the first and second light scattering elements 12 and 13 are selected to be between 1.5 to 2.5 times of the height of the pyramids of the second light scattering element 13. The height of the pyramids in the second light scattering element is typically in the range of 0.8 to 1.3 mm. Pyramids in both the first 12 and second 13 light scattering elements have an apex angle between about 85° to 95°.

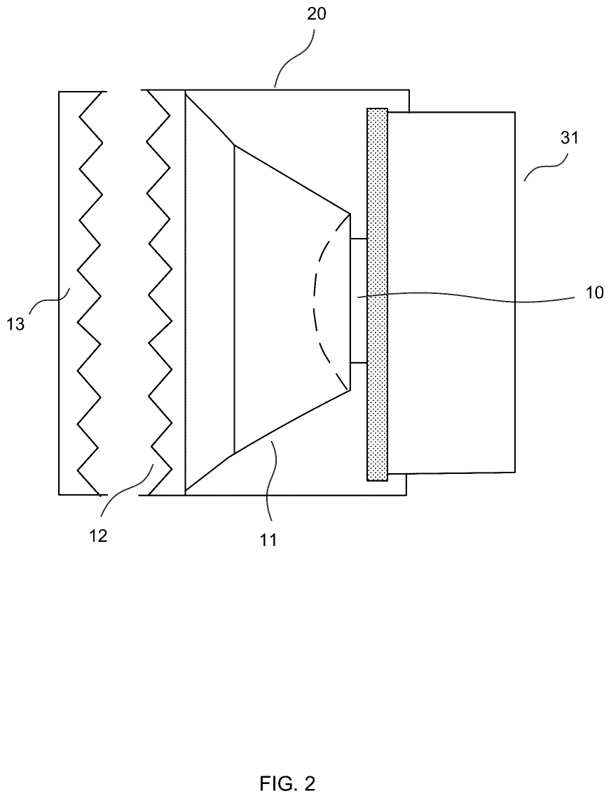

FIG. 2 shows the optical system 20 of FIG. 1 placed within a housing 31 that comprises the second scattering element 13. In examples where there is a plurality of optical systems within the housing 31, the second scattering element 13 may be sufficiently large to cover all optical systems. . . . In FIG. 2, the first and second scattering elements both comprise a plurality of pyramids which are arranged to form a raster. The housing comprises a head portion, the head comprising a cavity in which at least the optical system 20 may be held. As an example, the housing may comprise a wider head portion that has the hollow cavity, to surround the at least one optical system, as described in in EP 3 787 742 B1. However, the head portion of the common housing 31 of the present invention need not be wider than a body of the housing.

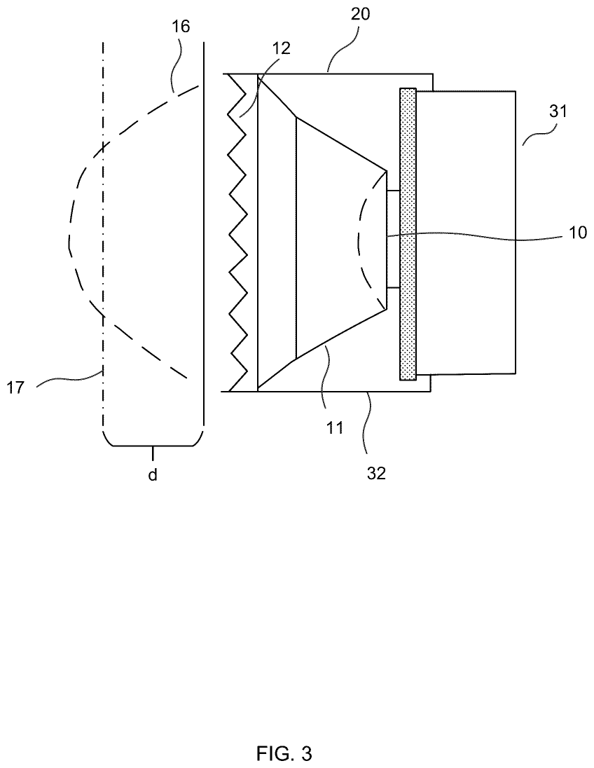

FIG. 3 is an exemplary figure showing the optical system 20 arranged as in FIG. 2 but where the second scattering element 13 is not used. In this exemplary figure, the second scattering element 13 is replaced by a planar, transparent closing element. In front of this closing element, semi-circle 16 drawn by a dashed line is shown, which illustrates the light intensity distribution curves measured in the plane of the drawing. Dot-dashed line 17 is drawn at a distance d from the rear face of the closing element and is parallel thereto. The light intensity of the light source 10, depicted by dashed line 16, at a distance greater than d from the rear face of the closing element is only higher than line 17 in a short discrete central region, and at all other regions it is much smaller. Optical systems with such sharply varying light distribution cannot be used for skin care, as in a large area under the line 17 the intensity is small and varies to a substantial extent.

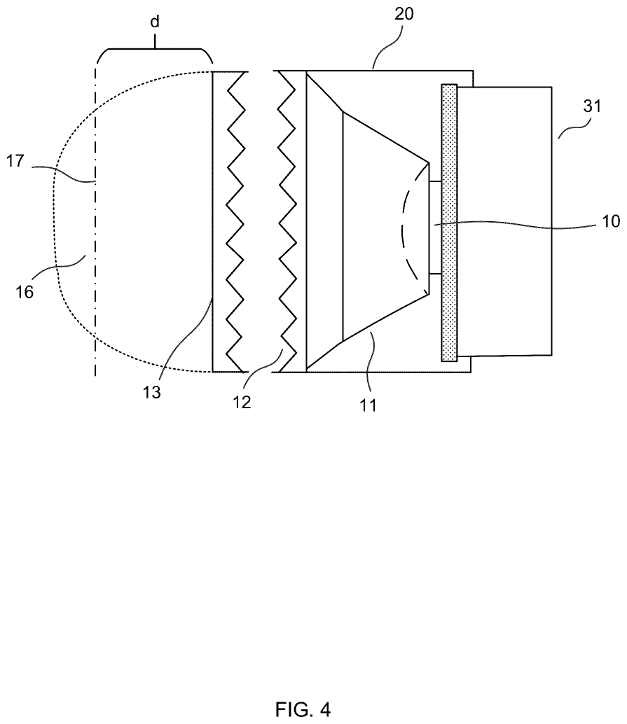

FIG. 4 illustrates an arrangement in accordance with the present invention. The system of FIG. 4 is similar to FIG. 3, with the addition of the second scattering element 13. As an effect of the multiple reflections that take place in the second scattering element 13, a very different light intensity curve 16 is obtained, which is more uniform and intensive above the whole area of the second light scattering element 13. In a much larger region than the system depicted in FIG. 3, the intensity is higher than at the reference line 17. Moreover, the light intensity is only slightly higher than this reference intensity across the length of the second scattering element, therefore this light distribution can be considered a substantially uniform. The size of this area above the line 17 is about the same as the diameter of the front part of the housing 32. As such, the area treated by the device will be uniformly illuminated, which is a significant difference compared to the arrangement without the second light scattering element 13.

In the sketches referred to herein, only the elevation view of the device has been illustrated. It will be readily understood by the person skilled in the art that the full or almost the full area behind the second light scattering element 13 and of the mouth of the frontal portion 32 of the housing can be uniformly illuminated.

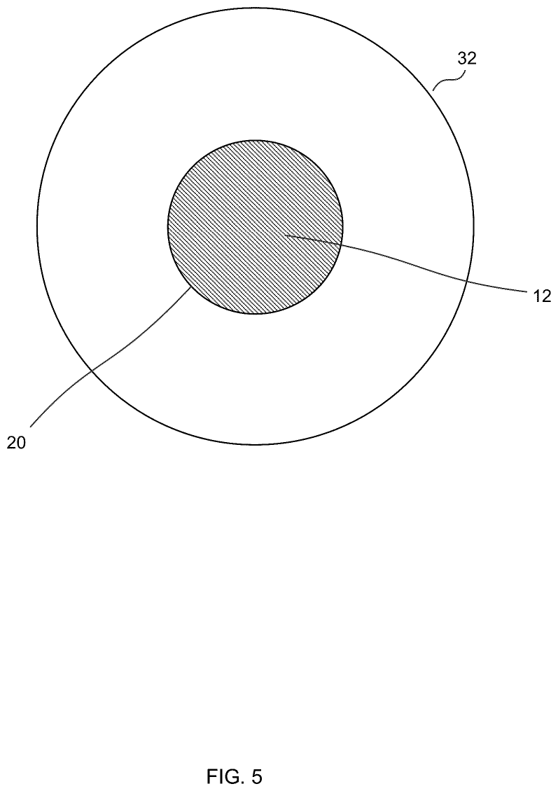

Reference is made now to FIG. 5 in which an optical system 20 is arranged centrally within the housing 32. In FIG. 5, the pyramids that constitute the first light scattering elements 12 are seen from their top surfaces. In examples where the laser light sources emit light in the infrared range of wavelengths (preferable at 808 nm or close thereto) which is not visible by the human eye, it is advantageous to place a light source emitting visible light, such as a light emitting diode (LED). For example, the optical system may comprise a visible light source adjacent to the laser light source 10. In examples where there are a plurality of laser light sources and respective first light scattering element 12 for each laser light source, a visible light source may be placed in a central opening among the optical systems between where the respective first light scattering elements abut. In examples where the first light scattering elements do not abut, visible light sources may be placed between adjacent first light scattering elements 12, which has the advantage of each visible light source indicating whether a respective laser light source is operating. The visible light source(s) 41 may be energized together with the laser light sources. Advantageously, the user can see when the device is turned on and is ready for treatment. The distribution shown in FIG. 4 is circularly symmetric if the four optical system are used.

A similar intensity distribution can be obtained if instead of using four optical systems only three of them is used in which their centers fall on the apex point of a regular triangle. Where there are three optical systems, the bodies of the three beam expanders may be made from a single piece. In this example, the three cones have a united common central portion. The forming of a common body for the three optical system can be made as illustrated in FIG. 2 of the previously cited EP 3 787 742 B1. Alternatively, the three beam expanders are three distinct pieces which are shaped to fit each other and are arranged adjacent to each other to form the same shape as the single piece. In both examples, the three optical beam expanders have a common central zone at the laser light source facing surface.

The first light scattering elements are also pyramids that are distributed in rows and columns along the whole frontal surface of the united optical systems.

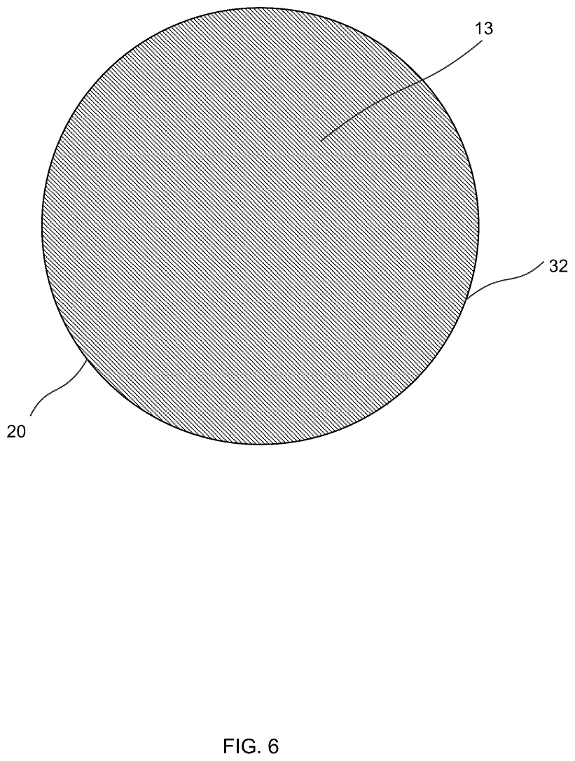

FIG. 6 shows the rear surface of the second light scattering element 13 with the pyramids arranged also in rows and columns. The pyramids can be prepared as extending out from the rear surface of the plastic material of the element 13 which is at the same time the frontal closing member of the housing 32 but can also be made as pyramid-shaped recesses or depressions made in the material of the element 13. The possibility of using inverse pyramids is also true for the first light scattering elements 12.

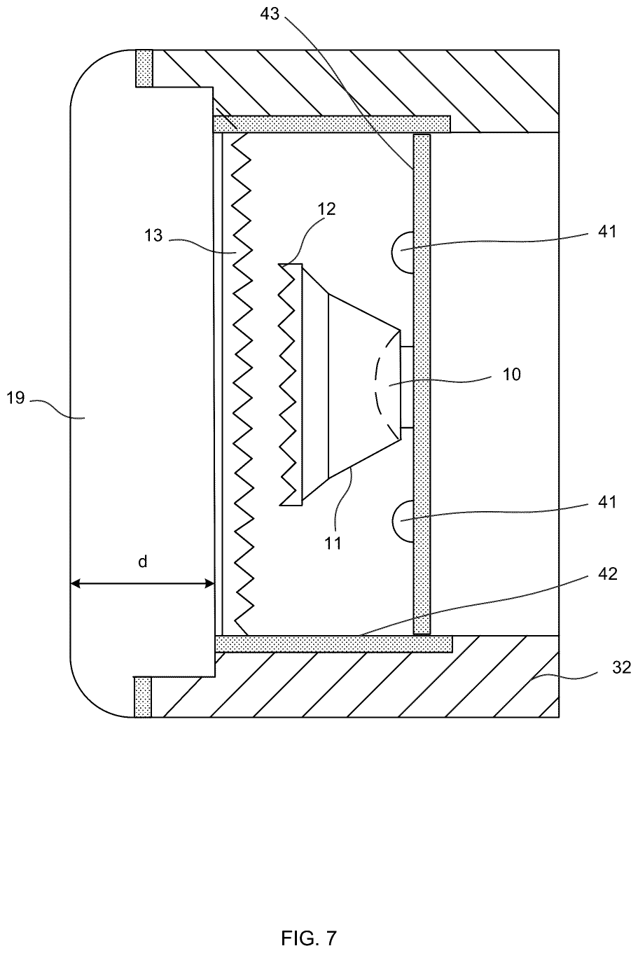

FIG. 7 shows the elevation sectional view of the embodiment shown in FIG. 4, in which the difference lies in that the second scattering element 13 and the transparent closing member 19 are made as one. The thickness is of the closing member in some examples is approximately the same distance as the distance d illustrated in FIG. 4, and therefore the full-frontal surface of the device (i.e., of the closing element 19) emits laser light with substantially the same intensity. During use, the circular front surface of the closing element 19 can be slightly contacted to the skin area to be treated. The light utilization efficiency is increased if the internal surfaces of the housing 32, like the cylindrical surface 42 around and in front of the optical systems and the planar rear surface 43 are covered with a light reflecting (mirroring) layer, since the light falling on such surfaces will also be reflected and utilized. Visible light sources 41 may also be placed adjacent to the laser light source, as described above.

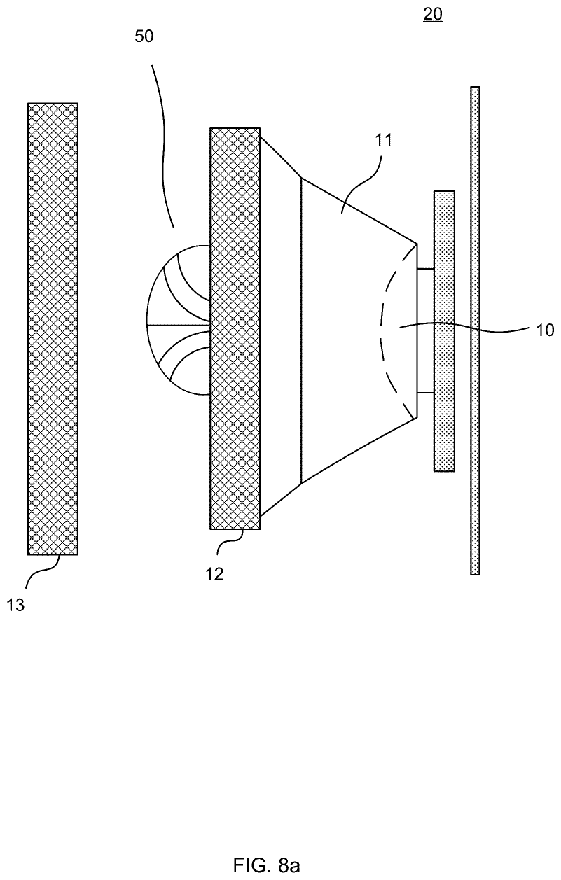

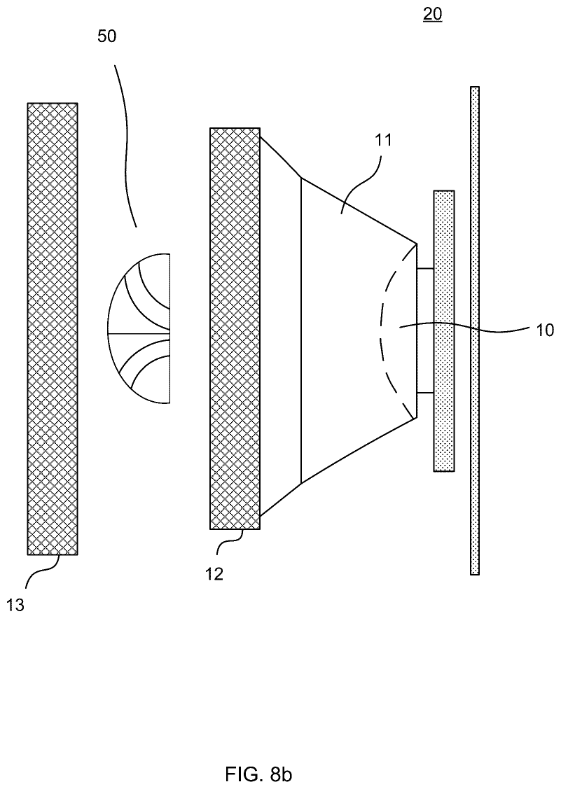

FIGS. 8a and 8b show an enlarged explanatory sketch of an optical system 20 used in the present invention in front of a single laser light source 10. In addition to the optical system 20 illustrated in FIG. 1, the optical system 20 of FIGS. 8a and 8b additionally comprise a fisheye lens 50 located between the first scattering element 12 and the second scattering element 13. In both FIGS. 8a and 8b, the fisheye lens 50 is positioned such that the wider base of the fisheye lens 50 faces the laser light source 10 and the first light scattering element 12 whilst the opposing, tapered end of the fisheye lens 50 faces the second light scattering element 13.

As depicted in FIG. 8a, the fisheye lens 50 may be located on the surface of the first light scattering element 12. For example, the fisheye lens 50 may be integral to the first scattering element 12 on a surface of the first scattering element 12 facing the second scattering element 13. Alternatively, the fisheye lens 50 may be placed in contact with the first light scattering element 12. For example, the fisheye lens 50 may be held in contact with the first light scattering element 12 using an adhesive.

However, as depicted in FIG. 8b, the fisheye lens 50 may be a separate element which is held between the first and second scattering elements 12, 13 at a distance between the first and second light scattering elements 12, 13. For example, the fisheye lens 50 may be held in place by one or more components within the housing 32 or by the housing 32 itself. The fisheye lens 50 may be placed between any of the first and second light scattering elements 12, 13 described herein.

The fisheye lens 50 further diffuses the laser light source 10. The fisheye lens is positioned such that it is in the path of a laser light source 10. Where there is a plurality of laser light sources, a fisheye lens may be placed in the light path of one or all of the respective laser light sources 10. Using this arrangement, the diffusion of the light is significantly increased whilst maintaining a great enough light intensity to treat human skin. The greater diffusion achieved by the fisheye lens further improves the uniformity of the light intensity of the laser light source 10 at a distance from the laser device resulting in a more even treatment area of the skin.

The material of the second scattering element 13, being part of the closing element 19 and of the two beam expanders 11, 21 should not affect the polarization of the emitted laser light, and such material is e.g., the previously mentioned polymethyl methacrylate.

The size of an embodiment, which is not a limiting example, can be preferably designed so that the diameter of the closing element 19 is around 40-50 mm. As another example, the distance d is around 8-12 mm. Three or four optical systems may be used and are arranged as described above. The laser light sources 10 emits light in the wavelength range which is optimum for the selected treatment, where 808 nm is a preferred wavelength. The power of the individual light sources is preferably between around 100 mW and 600 mW. Lasers of power greater than 600 mW may also be used. In case infrared light is used, which is not visible by the human eye, it is advantageous to use a pilot led light 41 which is turned on and off together with the laser lights.

The main field of use of the device is cosmetics but other kinds of uses can also be performed. As such, the cosmetic use of the low power therapy device according to any of the embodiments and examples described herein to treat skin is also provided.

The advantages of the present device include the uniform filed intensity and the high efficiency of the available light energy as well as the battery-operated use, whereby the device can be used anywhere and independent from the availability of mains supply, and there are no electrical shock hazards either.

In an example is provided a low power laser therapy device, which comprises: an optical system, wherein the optical system comprises: a laser light source; a beam expander in front of said light source; a raster arrangement of first light scattering elements on a frontal surface of the beam expander; and a housing around the optical system, wherein the housing comprises a mouth opening covered by a closing member, wherein, in front of the optical system, a raster arrangement of second light scattering element fills said mouth opening, and wherein the beam expander and the first and second light scattering elements are made from a non-polarizing transparent material.

In a further example, the optical system further comprises a fisheye lens positioned between the first scattering element and the second scattering element.

In a further example, the fisheye lens is integral to the first scattering element.

In a further example, said arrangement of the first and second light scattering element comprises a raster arrangement of pyramids arranged in rows and columns.

In a further example, said pyramids each have quadratic base and their apex angles is between 85° and 95°.

In a further example, the height of said pyramids is between 0.6 and 1.3 mm.

In a further example, the second light scattering element is integral to the closing member.

In a further example, the axial distance between the first and second light scattering elements is between 1.5 to 2.5 times of the height of the pyramids.

In a further example, the low power laser therapy device further comprises: a second optical system, a third optical system, wherein the three optical systems have circularly symmetrical shapes and their centre points fall to the apex point of a regular triangle.

In a further example, the three optical systems beam expanders comprise a common central portion and wherein the three optical systems comprise a common body.

In a further example, the low power laser therapy device further comprises: a third optical system; a fourth optical system, wherein the four optical systems have circularly symmetrical shapes and their centre points fall to the apex points of a square, and wherein adjacent beam expanders of the respective beam expanders of the four optical systems contact each other at respective single points.

In a further example, a material of the first and second light scattering elements and/or of the beam expanders is polymethyl methacrylate.

In a further example, an inner surface of the housing comprises a light reflecting layer.

In a further example, the laser light source is configured to emit light in an infrared range of wavelength, wherein the laser light source (10) is preferably configured to emit light at 808 nm.

In a further example, the low power laser therapy device comprises at least one LED light source emitting light in the visible range of wavelength and being switched together with said laser light sources and/or wherein the power of each one of the laser light sources (10) is between 100 mW and 600 mW.

1. A low power laser therapy device, which comprises:
an optical system, wherein the optical system comprises:
a laser light source;
a beam expander in front of said light source;
a raster arrangement of first light scattering elements on a frontal surface of the beam expander; and
a housing around the optical system,
wherein the housing comprises a mouth opening covered by a closing member,
wherein, in front of the optical system, a raster arrangement of second light scattering element fills said mouth opening, and
wherein the beam expander and the first and second light scattering elements are made from a non-polarizing transparent material.

2. The device according to claim 1, wherein the optical system further comprises a fisheye lens positioned between the first scattering element and the second scattering element.

3. The device according to claim 2, wherein the fisheye lens is integral to the first scattering element.

4. The device according to claim 1, wherein said arrangement of the first and second light scattering element comprises a raster arrangement of pyramids arranged in rows and columns.

5. The device according to claim 4, wherein said pyramids each have quadratic base and their apex angles is between 85° and 95°.

6. The device according to claim 4, wherein a height of said pyramids is between 0.6 and 1.3 mm.

7. The device according to claim 1, wherein the second light scattering element is integral to the closing member.

8. The device according to claim 4, wherein an axial distance between the first and second light scattering elements is between 1.5 to 2.5 times of a height of the pyramids.

9. The device according to claim 1, further comprising:
a second optical system,
a third optical system,
wherein the three optical systems have circularly symmetrical shapes and their centre points fall to an apex point of a regular triangle.

10. The device according to claim 9, wherein the three optical systems beam expanders comprise a common central portion and wherein the three optical systems comprise a common body.

11. The device according to claim 1, further comprising:
a third optical system;
a fourth optical system,
wherein the four optical systems have circularly symmetrical shapes and their centre points fall to an apex points of a square, and wherein adjacent beam expanders of the respective beam expanders of the four optical systems contact each other at respective single points.

12. The device according to claim 1, wherein a material of the first and second light scattering elements and/or of the beam expanders is polymethyl methacrylate.

13. The device according to claim 1, wherein an inner surface of the housing comprises a light reflecting layer.

14. The device according to claim 1, wherein the laser light source is configured to emit light in an infrared range of wavelength, wherein the laser light source is preferably configured to emit light at 808 nm.

15. The device according to claim 1, which comprises at least one LED light source emitting light in a visible range of wavelength and being switched together with said laser light sources and/or wherein the power of each one of the laser light sources (10) is between 100 mW and 600 mW.
